(19) United States
(12) Patent Application Publication
(10) Pub. No.: US 2020/0350049 A1
(43) Pub. Date: Nov. 5, 2020

(54) SYSTEMS AND METHODS FOR DISTRIBUTING CELL THERAPIES

(71) Applicant: Atara Biotherapeutics, Inc., South San Francisco, CA (US)

(72) Inventor: Derrell D. Porter, South San Francisco, CA (US)

(21) Appl. No.: 16/960,767

(22) PCT Filed: Jan. 7, 2019
(86) PCT No.: PCT/US19/12542

§ 371 Date: Jul. 8, 2020

Related U.S. Application Data
(60) Provisional application No. 62/614,834, filed on Jan. 8, 2018.

(51) Int. Cl. G16H 20/10; A61K 35/17; G06Q 10/08; G16H 10/60

(52) U.S. Cl. CPC G16H 20/10; G16H 10/60; G06Q 10/0832; A61K 35/17

(57) ABSTRACT

The disclosure relates to delivery systems, and corresponding methods, for selecting and delivering an allogeneic T-cell line for administration to a patient, e.g., according to the HLA profile of the patient's somatic or diseased cells.

### CROSS-REFERENCE TO RELATED APPLICATIONS

This application is filed as a National Stage of, and claims the benefit of priority under 35 USC 371, to PCT/US19/12542 filed Jan. 7, 2019, which claims the benefit of priority under 35 USC 119 to U.S. Provisional Application No. 62/614,834, filed Jan. 8, 2018, the entire contents of each are hereby incorporated by reference.

### FIELD OF THE DISCLOSED SUBJECT MATTER

The disclosed subject matter relates to a system, and corresponding method, for distributing cell therapies. Particularly, the presently disclosed subject matter is directed to an allogeneic T-cell therapy delivery system for selecting and delivering an allogeneic T-cell line for administration to a patient in need of allogeneic T-cell therapy.

### BACKGROUND

Allogeneic T-cell therapies are a promising class of immunotherapies for treating a range of disorders from cancer and viral infections to multiple sclerosis and post-transplant lymphoproliferative disorders. As compared to autologous T-cell therapies, which require obtaining and culturing a patient's T-cells before the therapy can be prepared and administered, allogeneic therapies offer the convenience of an off-the-shelf therapy. However, unlike other immunotherapies, such as anti-PD1 antibodies, allogeneic T-cell therapies require coordinating characteristics of the T-cell therapy with each patient's immune system to achieve desired levels of efficacy. This coordination, together with the complexities of preparing, characterizing, and storing the T-cell therapies, presents challenges to treating patients that are vastly different from traditional therapies that can simply be stocked at a pharmacy and distributed by a pharmacist based on a physician's prescription. While certain autologous T-cell therapies are known, e.g., CAR-T therapy, there are no commercially approved allogeneic T-cell therapies. Thus, there remains a need for the novel systems and methods for coordinating allogeneic T-cell therapies disclosed herein.

### SUMMARY OF THE DISCLOSED SUBJECT MATTER

The disclosed subject matter includes allogeneic T-cell therapy delivery systems for selecting and delivering an allogeneic T-cell line for administration to a patient in need of allogeneic T-cell therapy.

Such systems typically comprise a product repository including a plurality of samples of antigen-specific Cytotoxic T-Lymphocytes (CTLs), the CTLs of each sample having a known Human Leukocyte Antigen (HLA) profile and a known HLA restriction for a target antigen. In some embodiments, the antigen is a viral antigen, including at least one of: Human papilloma virus, Cytomegalovirus, BK virus, Epstein-Barr virus, John Cunningham (JC) virus, and/or Merkel cell virus (MCV); in other embodiments, the antigen is a Wilm's Tumor 1 (WT1) antigen. The CTLs in the product repository may be stored in unit-dose containers, such as single-use vials, or in any other suitable containers. To store the CTLs, the product repository may be maintained at very low temperatures, e.g., approximately −190° C. to −200° C. The CTLs may be disposed in any suitable order or arrangement, e.g., sorted by donor source and/or by donor source HLA type. In certain embodiments, the product repository lacks CTLs having a predetermined HLA characteristic. In certain preferred embodiments, the antigen-specific CTLs of the product repository meet a threshold for reactivity against cells (e.g., tumor cells) expressing the antigen. Preferably, CTLs of the product repository are free of pathogenic contaminants. The delivery systems comprise a communication channel, the communication channel receiving patient-characteristic data, including patient identification information and the HLA profile of the patient's somatic or diseased cells and a physician assent to treatment of the patient.

The HLA profile of the patient's diseased cells is preferable in most cases, as this profile is indicative of the cells to be targeted by the CTL therapy. However, in some cases, e.g., where the diseased cells are expected to share most or all of the HLA profile of the patient's somatic cells, the HLA profile of the patient's somatic cells may substitute for the HLA profile of the patient's diseased cells.

In some embodiments, the physician assent to treatment includes submission of a prescription or other physician-initiated request for allogeneic T-cell therapy for the patient. The assent may be received in advance of the patient's HLA profile information, simultaneously with the patient's HLA profile information, or subsequent to the patient's HLA profile information. In some embodiments, the patient-characteristic data includes transplant history, high resolution HLA data, weight, an HLA profile of cells from a transplanted organ, and/or an HLA profile of transplanted allogeneic hematopoietic cells.

The delivery systems also include an allogeneic T-cell match generator that determines an ordered set of cell lines from the product repository, the ordered set of cell lines prioritized at least according to a pre-determined match level between the HLA profile of the patient's somatic or diseased cells and HLA profiles and known restriction(s) of cell lines in the product repository. In some embodiments, the allogeneic T-cell match generator determines an ordered set of up to four prioritized cell lines. In certain embodiments, the ordered set of cell lines consists of a single cell line. In some embodiments, the first cell line in the ordered set of cell lines has two or more HLA profile matches with the patient's somatic or diseased cell HLA profile.

In some embodiments, the delivery systems communicate a first cell line of the ordered set of cell lines to the patient's physician, e.g., for the physician's approval prior to transport of the first cell line.

The delivery systems further comprise a registration module, the registration module receiving and registering the physician's assent to treatment of the patient and registering the first cell line for administering to the patient. In some embodiments, the allogeneic T-cell match generator and/or the registration module stores the ordered set of cell lines in association with the patient identification information. For example, the registration module can help the system ensure that the patient's treatment has been properly ordered by a physician, store the ordered set of cell lines for the patient for future reference, and/or track the patient's responses to the cell lines to ensure a suitable course of therapy is provided to the patient.

The delivery systems may further comprise a shipping module that coordinates transport of the first cell line, e.g., from the product repository, for administration to the patient. In some embodiments, the system, e.g., the allogeneic T-cell match generator and/or the shipping module, confirms current availability of the first cell line of the ordered set of cell lines in the product repository prior to communicating the first cell line to the patient's physician or coordinating transport of the first cell line. If the first cell line is not present in the product repository, the system can confirm current availability of the second cell line in the ordered set of cell lines, etc., until a suitable match is confirmed to be present in the product repository. Then that (presently inventoried) cell line can be communicated to the patient's physician and/or shipped for administration to the patient. The first such cell line administered to the patient will be considered the “first” cell line for purposes of the feedback module discussed below.

Because a course of therapy with a particular cell line may involve multiple administrations, the system may require multiple doses of a cell line to be present and available (e.g., unreserved) in the repository for that cell line to be considered available for a new patient. In such embodiments, in conjunction with communicating or shipping the first dose of a cell line to the patient, the system may reserve additional doses of that cell line for administration to that patient (e.g., 2-5 doses, preferably 3 or 4) in the event that a positive response is observed when that cell line is administered to the patient. If, as treatment progresses, the system determines that the patient should not receive additional doses of that cell line and/or should start receiving a different cell line, any reserved doses of the prior cell line may be returned to an available/unreserved status. Thus, this process for determining availability and reserving doses of a cell line may be performed each time a new cell line (e.g., the first cell line and any subsequent cell lines) are communicated/shipped as a part of a patient's treatment.

In some embodiments, the first cell line is shipped or transported to the physician within approximately 7 days, preferably within approximately 6 days, and even more preferably within approximately 5 days after receiving the physician's assent to treatment and/or the patient-characteristic data.

In some embodiments, the systems further comprise a source repository, the source repository including donor peripheral blood mononuclear cells.

In some embodiments, the systems further comprise a T-cell target activation module, wherein the T-cell target activation module activates T-cells by contacting T-cells with an antigen of interest to generate antigen-specific CTLs and populates the product repository with the antigen-specific CTLs.

In some embodiments, the delivery systems further comprise a feedback module, the feedback module receiving information representative of the effect of administering the first cell line to the patient. The feedback module may use this information to select further cycles of treatment for the patient. For example, when the feedback module receives a complete response or a partial response, the system may initiate delivery of a second cycle of the first cell line for administration to the patient. After receiving a second complete response or a third partial response, the system may render a treatment complete status. Similarly, when the feedback module receives a stable disease response, the system may initiate delivery of another cycle of the first cell line for administration to the patient. However, if the feedback module receives a progressive disease response or a stable disease response, the system may select a second cell line from the ordered set of cell lines for administering to the patient.

In embodiments wherein cell lines are communicated to the physician for approval, system may communicate the second cell line to the physician for approval prior to shipping. Similarly, if the feedback module receives two consecutive stable disease responses, the system may select a second cell line from the ordered set of cell lines for administration to the patient (optionally communicating this second cell line to the physician for approval prior to shipping). In some embodiments, the system confirms availability of the second cell line of the ordered set of cell lines in the product repository prior to communicating and/or shipping the second cell line. If administration of each cell line from the ordered set of cell lines returns a stable disease response or progressive disease response, the feedback module may render a treatment complete status or otherwise stop identifying and/or shipping cell lines for administration to the patient. The system may also store correlations between a patient's patient profile information, cell lines administered to that patient, and the patient response to that treatment. This information may be suitably aggregated and used to refine the T-cell match generation algorithm.

In some embodiments, the delivery systems identify cell lines receiving at least a partial response from the feedback module, and output a notification to replenish supplies of the identified cell lines when the product repository inventory of the identified cell lines falls below a threshold level. For example, the system may keep a list of cell lines whose inventory falls below a threshold level, and may output a notification if one of these cell lines receives a partial or complete response from the feedback module. Alternatively, the system may check the inventory of each cell line that receives a partial or complete response from the feedback module, and output a notification if the inventory is below a threshold level.

In some embodiments, the system is configured for implementation with patients at risk, or exhibiting symptoms, of Post-Transplant Lymphoproliferative Disorder (PTLD), e.g., PTLD associated with a prior solid organ transplant in the patient, or a prior hematopoietic cell transplant in the patient.

In some embodiments, the system is configured for implementation with patients at risk, or exhibiting symptoms, of multiple sclerosis.

In some embodiments, the system is configured for implementation with patients at risk, or exhibiting symptoms, of Cytomegalovirus infection.

In some embodiments, the system is configured for implementation with patients at risk, or exhibiting symptoms, of leukemia or solid tumor cancers.

In certain embodiments, the system includes a temperature sensor that monitors the temperature of the cell line(s) during transport of the cell line(s). In certain such embodiments, the system generates a warning if the temperature of the cell line(s) exceeds a threshold temperature (or exceeds a threshold temperature for a predetermined duration or longer) during transport. The warning can be communicated to the physician or other recipient to indicate that the cell line(s) should be discarded or otherwise not administered to the patient, or that the cell line(s) should be tested for quality assurance prior to administration.

In certain embodiments, the system may comprise a payment module, e.g., that initiates payment for the cell line(s) upon receipt of the shipment by the physician.

In some embodiments, an order (e.g., a prescription or other physician-initiated request) of the first cell line initiates shipment of three cycles of the cell line to the physician. For instance, the system may direct shipment of a second cycle of a first cell line approximately 7 days after the first cycle of the first cell line is shipped, e.g., so that the physician can administer the second cycle at an appropriate interval after the first cycle. Likewise, the system may direct shipment of a third cycle of a first cell line approximately 7 days after the second cycle of the first cell line is shipped, e.g., so that the physician can administer the third cycle at an appropriate interval after the second cycle. In certain such embodiments, the physician can direct the system to vary the shipment schedule for one or more of the cycles, e.g., to accommodate holidays, vacation schedules, or other events that may impact the ability to administer a cycle at a particular interval from the previous cycle.

In accordance with another aspect, the disclosed subject matter includes methods of providing a cell therapy to a patient in need of such therapy. Such methods typically comprise providing a product repository including a plurality of samples comprising antigen-specific Cytotoxic T-Lymphocytes (CTLs), the CTLs of each sample having a known Human Leukocyte Antigen (HLA) profile and a known HLA restriction for a target antigen. In some embodiments, the antigen is a viral antigen, including at least one of: Human papilloma virus, Cytomegalovirus, BK virus, Epstein-Barr virus, John Cunningham (JC) virus, and/or Merkel cell virus (MCV); in other embodiments, the antigen is a Wilm's Tumor 1 (WT1) antigen.

The methods include receiving patient-characteristic data, including patient identification information and an HLA profile of the patient's somatic or diseased cells. As noted above, the HLA profile of the patient's diseased cells is preferable in most cases, as this profile is indicative of the cells to be targeted by the CTL therapy. However, in some cases, e.g., where the diseased cells are expected to share most or all of the HLA profile of the patient's somatic cells, the HLA profile of the patient's somatic cells may substitute for the HLA profile of the patient's diseased cells.

An allogeneic T-cell match selection is generated by determining an ordered set of cell lines from the product repository, the ordered set of cell lines prioritized at least according to a pre-determined match level between the HLA profile of the patient's somatic or diseased cells and HLA profiles and known restriction(s) of cell lines in the product repository. In some embodiments, the an ordered set of up to four prioritized cell lines is generated. In certain embodiments, the ordered set of cell lines consists of a single cell line.

The method further includes receiving the physician's assent to treatment of the patient. This physician assent can be received before or after generating the T-cell match selection of orders set of cell lines. In some embodiments, the physician assent includes submission of a prescription or other physician-initiated request for allogeneic T-cell therapy for the patient. Further, the assent may be received in advance of the patient's HLA profile information, simultaneously with the patient's HLA profile information, or subsequent to the patient's HLA profile information. In addition to or in lieu of physician assent, the methods can also include communicating a cell line(s) for the physician's approval prior to transporting the first cell line.

In some embodiments, the method further includes storing the ordered set of cell lines in association with the patient identification information. This can help ensure that the patient's treatment has been properly ordered by a physician, store the ordered set of cell lines for the patient for future reference, and/or track the patient's responses to the cell lines to ensure a suitable course of therapy is provided to the patient.

The methods further include transporting the first cell line, e.g., from the product repository, for administration to the patient within approximately 7 days after receiving the physician's assent to treatment of the patient, within approximately 7 days after receiving the patient characteristic data, or within approximately 7 days after receiving the physician's approval of the first cell line.

In some embodiments, the current availability of the first cell line of the ordered set of cell lines is confirmed to be within in the product repository inventory prior to communicating the first cell line to the patient's physician or coordinating transport of the first cell line. If the first cell line is not present in the product repository, the method can confirm current availability of the second cell line in the ordered set of cell lines, etc., until a suitable match is confirmed to be present in the product repository. Then that cell line can be communicated to the patient's physician and/or transported for administration to the patient.

Additionally, the method also includes a source repository including donor peripheral blood mononuclear cells, contacting the T lymphocytes of these cells from the source repository with an antigen to form antigen-specific CTLs, and storing the antigen-specific CTLs within the product repository. Moreover, in certain embodiments the method includes expanding the antigen-specific CTLs; assessing allo-reactivity of the antigen-specific CTLs and excluding or discarding CTLs exhibiting allo-reactivity above a predetermined threshold prior to adding the cells to the product repository; and/or assessing anti-antigen reactivity of the antigen-specific CTLs and adding them to the product repository if the CTLs are determined to meet a threshold for anti-antigen reactivity. Similarly, the method can further include evaluating the HLA profile of the antigen-specific CTLs of the product repository and storing the HLA profile in connection with each sample of CTLs.

In some embodiments, the method further includes assessing the effect of the first cell line selection, wherein assessing includes: receiving disease response feedback from the physician; initiating a second cycle of the first cell line when at least a partial response is provided; or providing a next sequential cell line from the ordered set of cell lines for administration to the patient, and optionally communicating the next sequential cell line for physician approval.

The methods disclosed herein have a variety of applications, examples of which include administering the cell therapy to a patient at risk, or exhibiting symptoms, of Post-Transplant Lymphoproliferative Disorder (PTLD), multiple sclerosis, leukemia, or a solid tumor cancer, CMV infection, and/or a hematological disorder characterized by expression of a Wilm's Tumor 1 antigen.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic representation of the process of preparing antigen-specific CTLs in accordance with the disclosed subject matter.

FIGS. 2 and 3 are exemplary flow charts in accordance with the disclosed subject matter.

FIGS. 4 and 5 are process diagrams of an exemplary method of providing an allogeneic T-cell therapy in accordance with the disclosed subject matter.

### DETAILED DESCRIPTION

Reference will now be made in detail to exemplary embodiments of the disclosed subject matter, an example of which is illustrated in the accompanying drawings. The method and corresponding steps of the disclosed subject matter will be described in conjunction with the detailed description of the system.

Human leukocyte antigens (HLAs) are a group of normal proteins that play an important role in the way the immune system works. There are many different types of HLA proteins, some of which are found on the surface of nearly every cell in the human body, and each person has a characteristic combination of HLAs that contribute to their individual immune profile (also called an HLA genotype). One of the important functions of HLA proteins is that they present disease-related antigens to T-cells of the immune system. Each T-cell has a specific target antigen it is able to recognize, and it can only do so when its target antigen is presented by (i.e., joined to) an HLA protein. The HLA protein through which a Cytotoxic T-Lymphocyte (CTL) recognizes its target antigen and eliminates a diseased cell is known as its HLA restriction.

Various techniques are available for selecting an allogeneic T-cell line for therapeutic administration to a patient. See, for example, WO2016/073550, the entire contents of which are hereby incorporated by reference for the selection methods disclosed therein.

As described herein, a library (or “repository” as used interchangeably throughout) is incorporated which is composed of fully HLA-characterized CTL lines, with a breadth of HLA profiles and HLA restrictions. When a patient is in need of treatment, a novel CTL selection algorithm identifies the most appropriate CTL line matched to the patient's HLA genotype. In representative embodiments, once the line is selected:
1. The CTLs are thawed and administered intravenously over about 5 minutes in an outpatient or inpatient setting;
2. The administered CTLs circulate throughout the body, ignoring healthy cells that don't express the target antigen, and specifically identifying diseased cells that do express the antigen;
3. CTLs eliminate diseased cells when they recognize the target antigen connected to their particular HLA restriction; and
4. CTLs also undergo target-controlled proliferation. This means they expand in number as long as they encounter the target antigen. Once they no longer encounter the target antigen, proliferation of the CTLs stops and their numbers recede.

In accordance with an aspect of the present disclosure, the systems described herein provide allogeneic CTL technology to a patient suffering from cancer, certain autoimmune diseases, or a viral infection.

In the presently described systems, a matching algorithm identifies CTL lines in the library suitable for administration to any individual patient, based on matching certain key immune characteristics of the CTL lines to the unique immune profile of each patient. CTLs can be targeted for treating different diseases, for example, by:
Creating CTLs broadly targeted to recognize, for example, Epstein Barr Virus (EBV) and Cytomegalovirus (CMV) viral antigens, or an antigen associated with Wilms tumor 1 (WT1). Such CTLs can be deployed for treating hematologic tumors, solid tumors (e.g., nasopharyngeal carcinoma), and/or severe infectious diseases.
Using selective antigen targeting to create specifically targeted CTLs, e.g., that target specific EBV, CMV, Human papillomavirus (HPV), John Cunningham (JC) virus, Merkel cell virus (MCV) and/or BK viral antigens that are relevant to certain diseases. This selective antigen targeting can be utilized for treatment of certain solid tumors (e.g., gastric cancer) and autoimmune conditions (e.g., multiple sclerosis).

### CTL Manufacturing

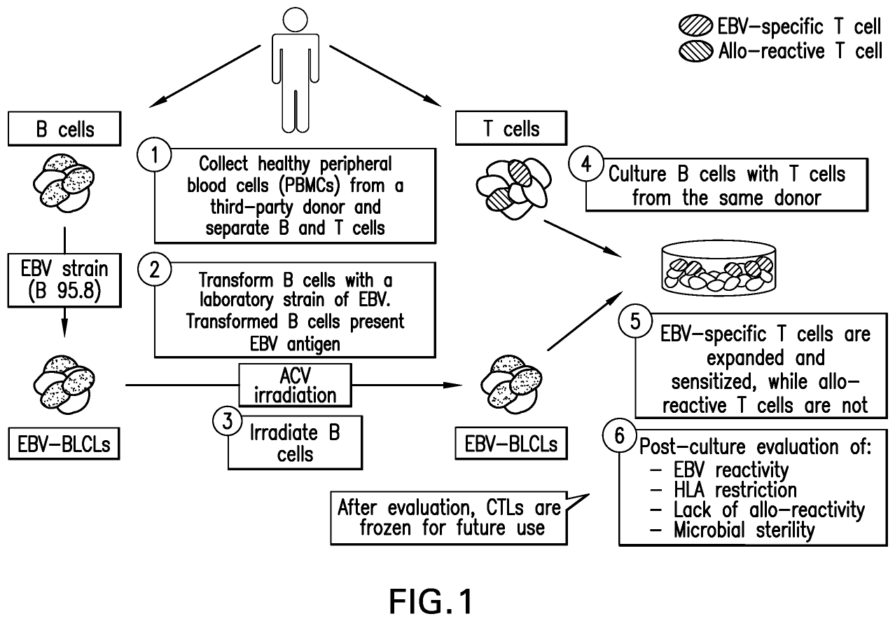

In accordance with an aspect of the disclosure, a CTL (cytotoxic T-cell) product library of inventory is created. As illustrated in FIGS. 1-3, the process starts with healthy white blood cells (peripheral blood mononuclear cells, or PBMCs) collected from a third-party donor. These donor cells are stored in a source repository (or “library” as used interchangeably herein). B-cells are separated and exposed to a target antigen (protein) of interest so they can present the antigen to T-cells. T-cells from the same third-party donor are then exposed to the antigen presenting B-cells. Only those T-cells with a receptor that recognizes the antigen of interest become activated to target diseased cells, and their numbers significantly expand with activation.

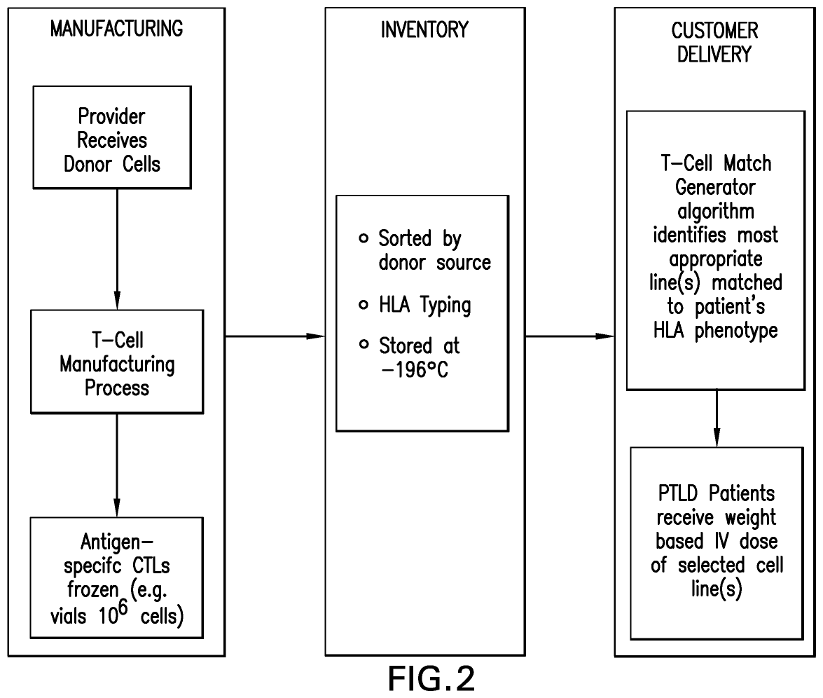

Those T-cells unable to recognize the antigen do not become activated and do not expand. The result is a CTL line enriched with cytotoxic T-cells specifically recognizing the target antigen. This CTL line is characterized by its human leukocyte antigen (HLA) and restriction profile and categorized in a product library of cryopreserved inventory. The product library comprises a diverse panel of ready-to-utilize CTL lines that allow suitable matching of CTL HLA profiles to each patient's individual immune profile. In some embodiments, the product repository lacks CTLs having a predetermined HLA characteristic. As is typically desired for infused therapies, the CTLs of the product repository are preferably free of pathogenic contaminants. An exemplary illustration of this process is depicted in FIG. 1, and a flow chart depicting the process is provided in FIG. 2.

As described herein, T lymphocytes of the allogeneic donor cells are contacted with an antigen to form antigen-specific CTLs, which are then stored within the product repository. The systems and methods of the present disclosure can be practiced with CTLs that target any of a variety of antigens. For example, the CTLs may target a viral antigen. Some non-limiting examples of such antigens include: antigen(s) from an Epstein-Barr Virus (EBV); antigen(s) from a Human Papilloma Virus (HPV); antigen(s) from a Cytomegalovirus; antigen(s) from a BK virus, antigen(s) from a John Cunningham (JC) virus, and/or antigen(s) from a Merkel cell virus (MCV). In other embodiments, the cells target a non-viral antigen, such as a Wilm's Tumor 1 (WT1) antigen.

Exemplary Embodiment

In an exemplary embodiment, the present disclosure includes an allogeneic T-cell therapy delivery system for selecting and delivering an allogeneic T-cell line for administration to a patient in need of allogeneic T-cell therapy. The system comprises a product repository which includes a plurality of samples comprising antigen-specific Cytotoxic T-Lymphocytes (CTLs). The CTLs of each sample have a known Human Leukocyte Antigen (HLA) profile and a known HLA restriction for the antigen.

The product repository containing antigen-specific CTLs can be configured as a single unit, or a series of repositories distributed geographically around a given territory. For instance, a single product repository can be centrally located, ideally equidistant, with respect to the largest organ transplant or other treatment centers throughout the United States. For example, for CTLs useful in treating PTLD, the repository can be located proximate those transplant centers which perform more complex procedures (e.g., heart and lung transplant), as these procedures typically are associated with an increased risk of Post-Transplant Lymphoproliferative Disease (PTLD). Alternatively, a plurality of product repositories can be employed, each containing an inventory of antigen-specific CTLs and located proximate to select treatment centers. Positioning of the repository proximate the treatment centers allows for rapid delivery of the selected cell line chosen from the repository, as is discussed in further detail herein.

The product repository may be maintained at ultra-low temperatures, e.g., approximately −190° C. to −200° C. In order to improve inventory control and facilitate more accurate matching to cell lines contained within, the product repository can be sorted by donor source and/or by donor source HLA type. To reduce shipment logistics and minimize delivery time, the CTLs in the product repository are preferably stored in single-use vials.

A communication channel is also included which receives patient-characteristic data, including patient identification information and the HLA profile of the patient's somatic or diseased cells. In some embodiments, the communication channel also receives (e.g., via a different transmission and/or timing than the patient identification information) a physician assent to treatment of the patient with the allogeneic T-cell therapy disclosed herein. The communication channel can be a dedicated and secure (e.g., encrypted) interface between health care providers (e.g., physicians) and the T-cell therapy provider (“Provider”). Patient information such as HLA profile, weight, medical history, transplant data (including HLA profile of cells from transplanted organ(s), and/or HLA profile of transplanted allogeneic hematopoietic cells), etc. can be inputted and/or transmitted by a heath care provider via the communication channel, which ensures compliance with the Health Insurance Portability and Accountability Act of 1996 (HIPPA). The communication channel also receives periodic patient outcomes of the treatment, as described in further detail herein.

In accordance with an aspect of the present disclosure, an allogeneic T-cell match generator is provided which determines an ordered set of cell lines from the product repository that are prioritized at least according to a pre-determined match level between the HLA profile of the patient's somatic or diseased cells and HLA profiles and known restriction(s) of cell lines in the product repository. The T-cell match generator employs an algorithm to prioritize, or rank, a plurality of cell lines, by applying various weighting factors and threshold values, to calculate the degree or match level between the HLA profile of the patient's somatic or diseased cells and HLA profiles and known restriction(s) of cell lines in the product repository.

The match level between the HLA profile and known restriction(s) of cell lines in the product repository can be based on a variety of parameters. For purpose of illustration and not limitation, an exemplary embodiment of the T-cell match generator algorithm selects CTL line(s) from the product repository that share two or more HLA allele matches with either the patient's diseased cells (e.g., if the HLA profile of the diseased cells is known) or the patient's somatic cells (e.g., if the HLA profile of the diseased cells is not known), and share an HLA restriction allele for at least one of the known viral epitopes.

In some embodiments, the T-cell match generator prioritizes a plurality of cell lines in a sequential order, with the first cell line having a greater match level (and thus greater likelihood of efficacy in vivo) than subsequent cell lines. The T-cell match generator disclosed herein can generate any desired number of matched cell lines; however, as each sequential cell line has a decreased expectation of efficacy, the exemplary embodiment depicted in the attached figures prioritizes four cell lines as optimal.

The allogeneic T-cell match generator also stores the ordered set of cell lines in association with the patient identification information. This allows for subsequent retrieval of the particular set of cell lines generated for a given patient for successive rounds of treatment, e.g., such that another shipment of a select cell line can be shipped, or so that the next sequential cell line from the previously generated set of cell lines can be shipped for administration to the patient. In other words, the T-cell match generator need not rerun the algorithm for the same patient every time a cell line is to be shipped to the patient (as described in further detail in the exemplary method of operation section herein). Storing the ordered set of cell lines in association with the patient identification information also allows for efficacy analysis of a given cell line based on patient outcome data, which can be fed back into the T-cell therapy system disclosed herein. This feedback loop can be factored into the algorithm generating prioritized cell lines, both for the current patient as well as future patients having similar histories/input data.

Alternatively, the system may simply identify the cell line with the best match available at that time and, after receiving feedback in the form of patient output data, either send an additional shipment of that cell line or re-run the algorithm and identify the best cell line available at that later time (which may be better, e.g., in the case of a cell line that was not available in the product repository at the time of the initial determination, or worse than the first cell line).

The T-cell match generator optionally communicates at least the first cell line of the ordered set of cell lines to the patient's physician. In some embodiments, only a single cell line is communicated to the physician at a given time; in other embodiments a plurality (e.g., all four) of matched cell lines can be communicated simultaneously to the physician.

Additionally, a registration module is included which registers the first cell line for administering to the patient. In some embodiments, the registration module also receives and registers the physician's assent to treatment of the patient. The physician assent to treatment can be indicated and registered in a variety of ways, e.g., submission of a paper or electronic prescription. Furthermore, the physician assent to treatment can be received in advance of the patient's HLA profile information, or simultaneously with the patient's HLA profile information. In some embodiments the physician's assent to treatment of the patient with the allogeneic T-cell therapy is received and registered prior to communicating any of the ordered cell lines outputted from the T-cell match generator. Indeed, the physician's assent can be a required input parameter, e.g., prerequisite, for initiating the T-cell match generator to output an ordered set of matched cell lines.

Alternatively, one (or all) of the ordered cell lines outputted from the T-cell match generator can be communicated to the physician for approval. This includes a higher level of review and consent from the physician than merely assenting to the treatment of the patient with allogeneic T-cell therapy described above. Here, the physician is notified of the specific cell line (and in some embodiments, the complete ordered set of all prioritized cell lines generated) and approves of the administration of that specific cell line (or complete ordered set) to a particular patient. If approval is not obtained for a particular cell line, the next sequential cell line can be communicated for approval.

In such embodiments, if the physician does not approve the ordered set of cell lines (whether a single cell line or plurality of cell lines are communicated), none of the cell lines are shipped. Conversely, when the physician does consent to the ordered set of cell lines, any subsequent doses of the first cell line or of sequential cell lines within the original ordered set can be shipped without further/supplemental physician approval.

In accordance with another aspect of the disclosure, a shipping module is included which coordinates transport of the first cell line for administration to the patient. The shipping module can arrange for release of a dose of the single cell line (or plurality of cell lines) from the product repository to a third-party courier for delivery of the cell line(s) to the physician. Alternatively, the shipping module can coordinate transport of the single cell line (or plurality of cell lines) directly from the product repository to the physician. Each vial(s), and/or each discrete shipment, can include a location tracking mechanism to provide continuous, real time location identification. This location tracking mechanism can be activated upon departure from the product repository and allows physicians, and the shipping entity, to ensure proper custody and control of the vial(s) throughout the transportation process. This tracking feature, along with the expedited delivery timeline (e.g., within 7 days as described further below) facilitate lean inventories and permit physicians greater flexibility and precision in scheduling patient infusions. Similarly, each vial(s) can include a tamper-evident feature to signal any contamination/disruptions which could adversely impact the integrity, safety, and/or efficacy of the cell line(s) contained therein.

The shipping module can provide and/or monitor a cold-chain delivery system in which the container(s) include a coolant (e.g., liquid nitrogen) or other temperature control system to maintain the cell lines(s) at a predetermined temperature such as at or below approximately −150° C., (until thawed for administration to the patient). The shipping container can also include a thermal indicator which provides a record of the temperature of the cell line(s) throughout the duration of transit. Additionally, the thermal indicator can include an alarm/indicator, e.g., that gives an alert or other signal in the event that a particular cell line experienced a temperature outside the range of approved temperatures or exceeded a temperature outside the range of approved temperatures for more than a predetermined length of time. If triggered, this alarm/indicator can alert the physician that the safety or efficacy of the cell line may have been compromised or that the quality of the cell line should be verified prior to administration.

In some embodiments, each individual dose of each cell line consists of approximately 2×10⁶ cell per kilogram of patient body weight. In some embodiments, each shipped vial of cells is a single use, cryopreserved vial that contains from approximately 1×10⁷ to approximately 6×10⁷ cells suspended and frozen in Dimethyl sulfoxide (DMSO), human serum albumin and buffered saline, with each vial remaining cryopreserved until thawed for administration to the patient.

In some embodiments, the first cell line is delivered to the physician within approximately 7 days after receiving the physician assent to treatment and/or patient characteristic data; within approximately 6 days after receiving the physician assent to treatment and/or patient characteristic data; or within approximately 5 days after receiving the physician assent to treatment and/or patient characteristic data; or within approximately 4 days after receiving the physician assent to treatment and/or patient characteristic data; or within approximately 3 days after receiving the physician assent to treatment and/or patient characteristic data.

In some embodiments, the first cell line is delivered to the physician within approximately 7 days after receiving the physician approval of the matched cell line(s); within approximately 6 days after receiving the physician approval of the matched cell line(s); or within approximately 5 days after receiving the physician approval of the matched cell line(s).

In some embodiments, the first cell line is delivered to the physician within approximately 7 days after the allogeneic T-cell match generator determines an ordered set of cell line(s); within approximately 6 days after the allogeneic T-cell match generator determines an ordered set of cell line(s); or within approximately 5 days after the allogeneic T-cell match generator determines an ordered set of cell line(s); or within approximately 4 days after the allogeneic T-cell match generator determines an ordered set of cell line(s); or within approximately 3 days after the allogeneic T-cell match generator determines an ordered set of cell line(s).

Treatment of a patient with a given cell line(s) can include a regimen of multiple (e.g., three) separate cycles, or doses, of that particular cell line to the patient, each following the prior dose/cycle by a predetermined time interval (e.g., approximately 7 days). In preferred embodiments, individual doses/cycles of a particular cell line are not shipped simultaneously. Instead, each dose/cycle of a particular cell line, which is approximately 2×10⁶ cell per kilogram of patient body weight, is shipped separately, e.g., at intervals calculated to coincide with the intended dosing frequency (e.g., approximately 7 days between shipment of cycles of the same cell line). This practice relieves any storage (within acceptable temperature range) burden on the physician, and helps ensure that successive cycles are of similar quality to the initial cycle. In some embodiments, the shipment module can default to automatically ship cycles of the chosen cell line to the physician at 7-day increments (i.e., 7 days between shipments). The shipment module can target shipments for transport and/or delivery on regular business days (e.g., to avoid holidays and weekends (when physician's offices may be closed or when transport may be interrupted), or to accommodate variations in a patient's schedule, etc.

Additionally, the shipment module may include the flexibility for the physician or other operator to adjust the shipment schedule for each does in response to physician input (e.g., through the communication channel). For example, a physician can determine that cycle number 2 (and/or cycle number 3) should deviate from the default schedule of 7 days between doses. Accordingly, the physician or other operator can direct the shipment module to schedule shipment and/or delivery of cycle number 2 (and/or cycle number 3) by one or two days, e.g., per the physician's discretion.

Additionally, the systems and methods disclosed herein can confirm current availability of a given (e.g., first) cell line of the ordered set of cell lines in the product repository prior to communicating or shipping the first cell line to the patient's physician. In some embodiments, confirming availability of the first cell line includes confirming the availability of at least three doses of a given (e.g., first) cell line, and the system reserves two additional doses of the first cell line in conjunction with shipping a dose of the first cell line to the patient's physician.

The systems disclosed herein may also provide a mechanism for confirmation of delivery of the shipment to the physician. In some embodiments, upon receipt of the cell line(s), the physician can send a confirmation, e.g., through the communication channel, verifying that the cell line(s) have been timely received (e.g. within a predetermined time interval as described above) and are in suitable condition for administering to the patient. The shipping module can include a return label/packaging for the physician to return the cell line(s) in the event that the cell line(s) do not meet quality standards (e.g., exceeded a temperature threshold during shipment) or the patient dies before receipt of the cell line(s). This return packaging can likewise maintain the cell line(s) at the predetermined temperature range so that the returned cell line(s) can be returned to the repository for future use.

Furthermore, the systems of the present disclosure may also include a payment module for coordinating payment to the allogeneic T-cell therapy provider. In some embodiments, a delivery confirmation by the physician or other recipient can trigger bill/invoice generation from the payment module. Alternatively, an invoice can be sent simultaneously with the cell line(s) shipment and conditioned for payment upon receipt. In certain such embodiments, the system can initiate processing of a payment upon delivery confirmation by the physician or other recipient.

Payment can be effected in a variety of ways, including via third-party vendors. The third-party vendor can be an intermediary that facilitates physician/patient access to the allogeneic T-cell therapy provider and does not assume title or possession of the cell line(s). Alternatively, in some embodiments, the third-party vendor does assume title to the cell line(s) once they are sold. In such scenarios, at the point of sale, e.g., once the cell line(s) are shipped and payment is made, the third-party vendor can assume “flash title” to the cell line(s)—i.e., the third-party vendor assumes legal title from the point of sale to the time the cell line(s) is delivered to the physician. An exemplary embodiment of a flash title model which can be employed with the present disclosure is disclosed in U.S. Pat. No. 7,860,757, the entirety of which is hereby incorporated by reference. Additionally, in some embodiments, a third-party vendor may submit payment to the allogeneic T-cell therapy provider and subsequently invoice either the physician or the patient's health care provider.

Exemplary Method of Operation

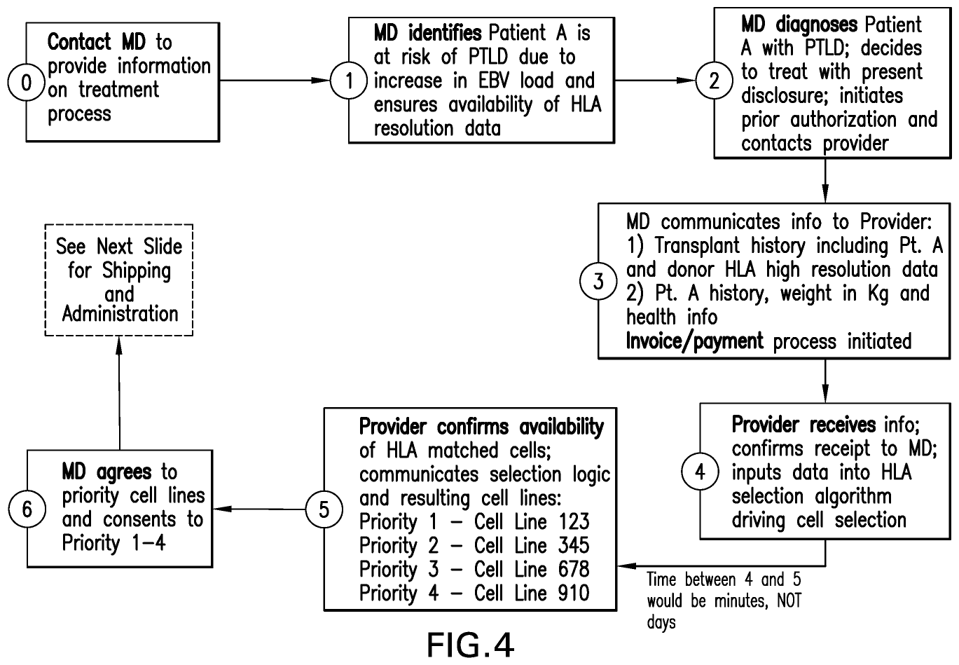

For purpose of illustration and not limitation, an exemplary method is described herein and illustrated in FIGS. 4-6.

A method of providing a cell therapy to a patient in need of such therapy in accordance with the present disclosure includes providing a product repository (formed and populated from the donor peripheral blood mononuclear cells of the source repository as described above) having a plurality of samples comprising antigen-specific Cytotoxic T-Lymphocytes (CTLs), the CTLs of each sample having a known Human Leukocyte Antigen (HLA) profile and a known HLA restriction for said antigen. In some scenarios, the provider will be the sole custodian of the source repository, product repository and all of the procedures (and requisite equipment) for transforming the cells from the source repository to the antigen-specific CTLs within the product repository. In other embodiments, the various components of the present disclosure can be owned or maintained by a plurality of discrete entities. For example, the provider can be responsible for all aspects leading up to shipment of the cell lines to the physician, with a third-party vendor taking responsibility for the logistics of delivery to the physician.

As shown in FIG. 4, step 0, a physician (labeled “MD” in the figure) is educated about the availability of the present system and method of cell therapy. The physician identifies a patient is at risk of a particular disease or disorder which is treatable with CTLs as described herein. (Note, the exemplary embodiment shown in the figures depicts the application for a person at risk of PTLD due to an increase in EBV load; however, it is to be understood that the present disclosure is equally applicable to additional diseases/disorders and/or risk identifiers where treatment with CTLs may be beneficial.)

Once it is determined (step 2 in FIG. 4) that the cell therapy of the present disclosure is desired, the physician (or healthcare provider) contacts the Provider to inquire about, and initiate, the instant cell therapy treatment. In some embodiments, the assent of the physician is required to initiate the allogeneic T-cell match generator. In such embodiments, this assent can be configured and/or memorialized as a physician's prescription. In some embodiments, specific physician assent is not required for each patient individually. For example, a physician can establish a prospective assent for a plurality of patients, allowing for immediate communication of the ordered set of cell lines upon input of the patient-characteristic data.

Next, patient-characteristic data, including patient identification information and an HLA profile of the patient's somatic or diseased cells is received via a communication channel by the provider (step 3 in FIG. 4). This patient identification information is inputted into the allogeneic T-cell match generator, which outputs an allogeneic T-cell match selection. As described above, the T-cell match selection includes an ordered set of cell lines from the product repository, which are prioritized according to a pre-determined match level between the HLA profile of the patient's somatic or diseased cells and HLA profiles and known restriction(s) of cell lines in the product repository.

In some embodiments, only the first cell line of the ordered set of cell lines outputted from the allogeneic T-cell match generator is communicated to the physician. In other embodiments, a plurality (or all) of the ordered set of cell lines are communicated. Additionally or alternatively, in some embodiments, the allogeneic T-cell match generator confirms current availability of the first cell line of the ordered set of cell lines in the product repository prior to communicating the first cell line to the patient's physician. In other words, if the first cell line outputted from the allogeneic T-cell match generator is not available, the system advances to the subsequent cell line of the ordered set of cell lines (until a cell line is identified which is presently within the product repository inventory) and communicates this subsequent cell line to the physician. Similarly, this inventory audit to determine if any particular cell line is available can be performed as a prerequisite to communicating any cell line to the physician, including subsequent orders of a particular cell line that was previously available. Of course, in systems that reserve subsequent doses of a particular cell line at the time of designating/communicating/shipping the first dose of a cell line, repeat checks for availability of that cell line should not be necessary.

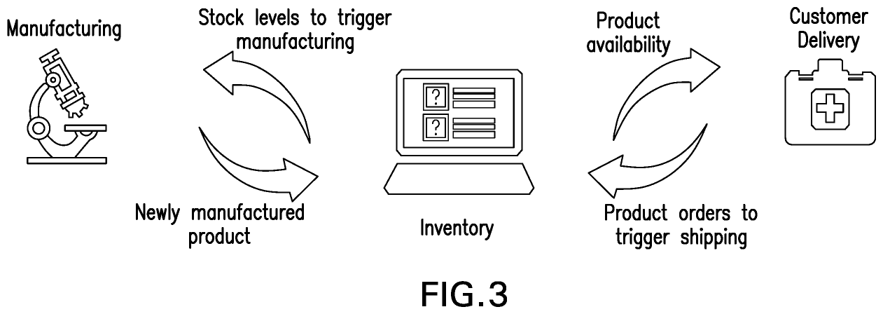

In those scenarios in which a particular cell line is not available within the product repository, the system selects the next sequential cell line (i.e., with the next highest match level) for communication to the physician. A graphical illustration of this product repository inventory audit is depicted in FIG. 3.

In some embodiments, and as shown as step 6 in FIG. 4, the physician's approval of the outputted cell line(s) is required in advance of shipment. In such embodiments, if the physician does not approve a first cell line, the subsequent cell line is communicated/proposed to the physician for approval (this procedure can iterate as required until a cell line is approved).

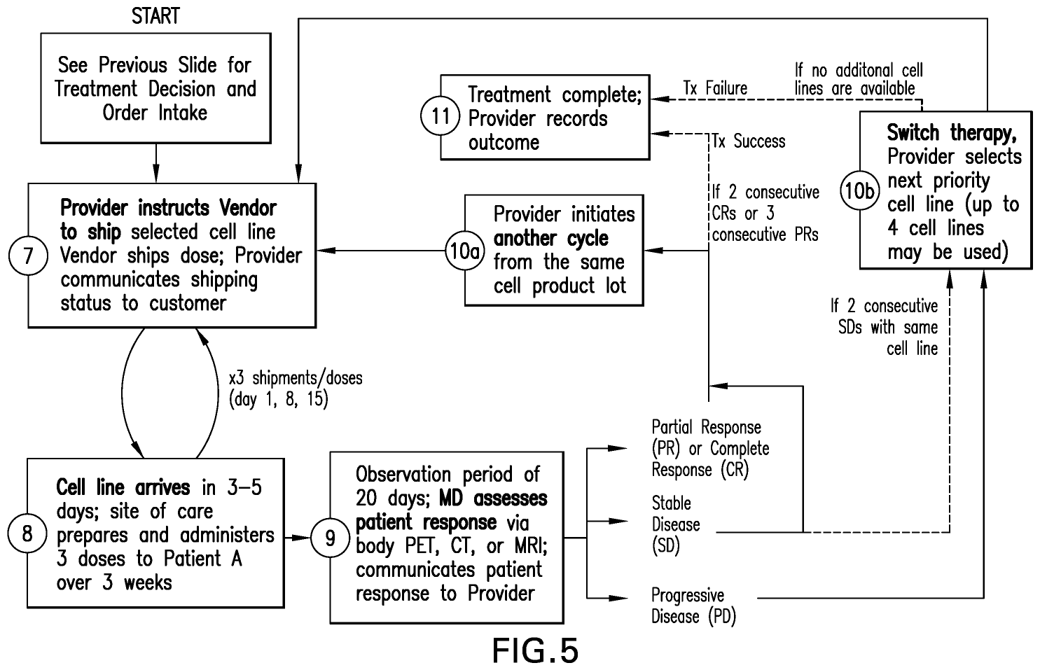

The selected cell line(s) is then prepared for shipment to the physician (step 7 in FIG. 5). In some instances, there may be a plurality or surplus of any particular cell line(s) within the product repository. Accordingly, in some embodiments, the system can evaluate which particular “batch” of a selected cell line(s) to ship based on a variety of parameters, such as expiration date, location and accessibility within the product repository, etc. As described in further detail above, the cell line(s) can be delivered within seven days to the physician for administration to the patient, an advantage over autologous systems that require time for a patient's cells to be processed into therapeutic CTLs that can be administered. The particular treatment regimen will vary dependent on patient needs, with an exemplary regimen shown in step 8 of FIG. 4 as being three doses over a three week interval.

In accordance with another aspect of the disclosure, a feedback module (steps 9-11 in FIG. 5) receives information representative of the effect of administering the first cell line (and any subsequent line(s)) to the patient. This feedback of cell line(s) efficacy can be inputted into the feedback module from the physician via the communication channel, optionally at predetermined intervals (e.g., daily, weekly, etc.), such as by prompting the physician to enter such feedback.

As shown in step 10a in FIG. 5, the system initiates delivery of a second cycle of the first cell line for administration to the patient when the feedback module receives a Partial Response or a Complete Response. Similarly, the system initiates delivery of another cycle of the first cell line for administration to the patient when the feedback module receives a Stable Disease Response. For embodiments of the present disclosure directed towards proliferative disorders, e.g., PTLD and cancer, this criteria can be quantified in accordance with the Response Evaluation Criteria In Solid Tumors (RECIST) standards, which, for target lesions, are as follows:
Complete Response (CR): Disappearance of all target lesions.
Partial Response (PR): At least a 30% decrease in the sum of the Longest Diameter (LD) of target lesions, taking as reference the baseline sum LD.
Stable Disease (SD): Neither sufficient shrinkage to qualify for PR nor sufficient increase to qualify for PD, taking as reference the smallest sum LD since the treatment started.
Progressive Disease (PD): At least a 20% increase in the sum of the LD of target lesions, taking as reference the smallest sum LD recorded since the treatment started or the appearance of one or more new lesions.

As shown in step 11 in FIG. 5, the system renders a treatment complete status after receipt of two consecutive Complete Responses from the feedback module, or three consecutive Partial Responses.

As shown in step 10b in FIG. 5, the system selects a second cell line (i.e., the next prioritized match) from the ordered set of cell lines for administering to the patient when the feedback module receives: i) a Progressive Disease response; ii) a Stable Disease response; or two consecutive Stable Disease responses from the same cell line. In some embodiments, where physician approval is required prior to each change in the CTL therapy, this second cell line is first communicated to the physician for approval before being prepared for shipment. Also, as described above, an inventory audit can be performed to confirm availability of this second cell line within the product repository prior to communicating with the physician. Conversely, in some embodiments, once physician assent is given, the selection of the first cell line outputted from the T-cell match generator, the decision to ship and administer more samples of the same first cell line, or the decision to switch to a different cell line can all be performed without obtaining or registering the physician's approval.

As shown in step 11 in FIG. 5, the feedback module renders a treatment complete status after administration of each cell line from the ordered set of cell lines returns a Stable Disease response or Progressive Disease response.

In some embodiments, the system identifies cell lines receiving a partial or complete response from the feedback module, and outputs a notification to replenish supplies of the identified cell lines when the product repository inventory of the identified cell lines falls below a threshold level. In some embodiments, the threshold level can be based on the number of vials of each cell line within the repository, e.g., 12, 15, 18, 21, 24, 27 or 30 vials. Additionally or alternatively, the replenishment notification can be triggered based on historical demand cycles, expiration dates of current inventories, geographical and/or demographic trends, forecasts, etc.

As described herein, the present disclosure has applications in numerous therapeutic treatment regimens. For example, the system and corresponding methods disclosed herein can be employed for administering the cell therapy to a patient at risk, or exhibiting symptoms, of any or all of the following: Post-Transplant Lymphoproliferative Disorder (PTLD); multiple sclerosis; leukemia, or a solid tumor cancer; BK virus, Cytomegalovirus (CMV), Epstein-Barr virus (EBV), Human papilloma virus (HPV), John Cunningham (JC) virus, and/or Merkel cell virus (MCV) infections; or a hematological disorder characterized by expression of a Wilm's Tumor 1 antigen.

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

Thus, the foregoing description of specific embodiments of the disclosed subject matter has been presented for purposes of illustration and description. It is not intended to be exhaustive or to limit the disclosed subject matter to those embodiments disclosed. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. An allogeneic T-cell therapy delivery system for selecting and delivering an allogeneic T-cell line for administration to a patient in need of allogeneic T-cell therapy, comprising:
a product repository, the product repository comprising a plurality of samples comprising antigen-specific Cytotoxic T-Lymphocytes (CTLs), the CTLs of each sample having a known Human Leukocyte Antigen (HLA) profile and a known HLA restriction for said antigen;
a communication channel, the communication channel receiving patient-characteristic data, including patient identification information and the HLA profile of the patient's somatic or diseased cells and, optionally, a physician assent to treatment of the patient with the allogeneic T-cell therapy;
an allogeneic T-cell match generator, wherein the allogeneic T-cell match generator:
determines an ordered set of cell lines from the product repository, the ordered set of cell lines prioritized at least according to a pre-determined match level between the HLA profile of the patient's somatic or diseased cells and HLA profiles and known restriction(s) of cell lines in the product repository;
a registration module, the registration module receiving and registering the first cell line for administering to the patient; and
a shipping module, the shipping module coordinating transport of the first cell line for administration to the patient.

2. The allogeneic T-cell therapy delivery system of claim 1, wherein the allogeneic T-cell match generator stores the ordered set of cell lines in association with the patient identification information.

3. The allogeneic T-cell therapy delivery system of any preceding claim, wherein the registration module receives and registers the physician assent to treatment of the patient.

4. The allogeneic T-cell therapy delivery system of claim 3, wherein the physician assent to treatment includes submission of a prescription.

5. The allogeneic T-cell therapy delivery system of claim 3 or 4, wherein the physician assent to treatment is received in advance of the patient's HLA profile information.

6. The allogeneic T-cell therapy delivery system of claim 3, 4 or 5, wherein the physician assent to treatment is received simultaneously with the patient's HLA profile information.

7. The allogeneic T-cell therapy delivery system of any preceding claim, wherein the first cell line of the ordered set of cell lines is communicated to the patient's physician for approval prior to transport of the first cell line.

8. The allogeneic T-cell therapy delivery system of any preceding claim, further comprising a source repository, the source repository including donor peripheral blood mononuclear cells.

9. The allogeneic T-cell therapy delivery system of any preceding claim, further comprising a T-cell target activation module, wherein the T-cell target activation module:
activates T-cells by contacting T-cells with an antigen of interest to generate antigen-specific CTLs; and
populates the product repository with the antigen-specific CTLs.

10. The allogeneic T-cell therapy delivery system of any preceding claim, wherein the system confirms current availability of the first cell line of the ordered set of cell lines in the product repository prior to communicating or shipping the first cell line to the patient's physician.

11. The allogeneic T-cell therapy delivery system of claim 10, wherein confirming availability of the first cell line comprises confirming availability of at least three doses of the first cell line, and the system reserves two additional doses of the first cell line in conjunction with shipping a first dose of the first cell line to the patient's physician.

12. The allogeneic T-cell therapy delivery system of any preceding claim, wherein the first cell line is shipped to the physician within approximately 7 days after receiving the physician assent to treatment and/or the patient characteristic data.

13. The allogeneic T-cell therapy delivery system of any preceding claim, wherein the first cell line is shipped to the physician within approximately 6 days after receiving the physician assent to treatment and/or the patient characteristic data.

14. The allogeneic T-cell therapy delivery system of any preceding claim, wherein the first cell line is shipped to the physician within approximately 5 days after receiving the physician assent to treatment and/or the patient characteristic data.

15. The allogeneic T-cell therapy delivery system of any preceding claim, wherein the first cell line is shipped to the physician within approximately 4 days after receiving the physician assent to treatment and/or patient characteristic data.

16. The allogeneic T-cell therapy delivery system of any preceding claim, wherein the first cell line is shipped to the physician within approximately 3 days after receiving the physician assent to treatment and/or patient characteristic data.

17. The allogeneic T-cell therapy delivery system of any preceding claim, wherein the first cell line is administered to the patient within approximately 7 days after receiving the physician assent to treatment and/or patient characteristic data.

18. The allogeneic T-cell therapy delivery system of any preceding claim, wherein the first cell line is administered to the patient within approximately 6 days after receiving the physician assent to treatment and/or patient characteristic data.

19. The allogeneic T-cell therapy delivery system of any preceding claim, wherein the first cell line is administered to the patient within approximately 5 days after receiving the physician assent to treatment and/or patient characteristic data.

20. The allogeneic T-cell therapy delivery system of any preceding claim, wherein the first cell line is administered to the patient within approximately 4 days after receiving the physician assent to treatment and/or patient characteristic data.

21. The allogeneic T-cell therapy delivery system of any preceding claim, wherein the first cell line is administered to the patient within approximately 3 days after receiving the physician assent to treatment and/or patient characteristic data.

22. The allogeneic T-cell therapy delivery system of claim 7, wherein the first cell line is shipped to the physician within approximately 7 days after receiving the physician's approval.

23. The allogeneic T-cell therapy delivery system of claim 7, wherein the first cell line is shipped to the physician within approximately 6 days after receiving the physician's approval.

24. The allogeneic T-cell therapy delivery system of claim 7, wherein the first cell line is shipped to the physician within approximately 5 days after receiving the physician's approval.

25. The allogeneic T-cell therapy delivery system of any preceding claim, wherein shipment to the physician, and/or administration to the patient, of the first cell line includes a first dose of the first cell line.

26. The allogeneic T-cell therapy delivery system of any preceding claim, further comprising a feedback module, the feedback module receiving information representative of the effect of administering the first cell line to the patient.

27. The allogeneic T-cell therapy delivery system of claim 26, wherein the system initiates delivery of a second cycle of the first cell line for administration to the patient when the feedback module receives a partial response.

28. The allogeneic T-cell therapy delivery system of any one of claims 26-27, wherein the system initiates delivery of another cycle of the first cell line for administration to the patient when the feedback module receives a complete response.

29. The allogeneic T-cell therapy delivery system of any one of claims 26-28, wherein the system initiates delivery of another cycle of the first cell line for administration to the patient when the feedback module receives a stable disease response.

30. The allogeneic T-cell therapy delivery system of any one of claims 27-29, wherein initiating another cycle of the first cell line includes initiating a second dose.

31. The allogeneic T-cell therapy delivery system of any one of claims 26-30, wherein the system renders a treatment complete status after receipt of two consecutive complete responses.

32. The allogeneic T-cell therapy delivery system of claim 31, wherein the system releases any doses reserved for the patient when rendering the treatment complete status.

33. The allogeneic T-cell therapy delivery system of any one of claims 26-32, wherein the system renders a treatment complete status when the feedback module receives three consecutive partial responses.

34. The allogeneic T-cell therapy delivery system of any one of claims 26-33, wherein the system selects a second cell line from the ordered set of cell lines for administering to the patient when the feedback module receives a progressive disease response or a stable disease response, and optionally communicates the second cell line for physician approval.

35. The allogeneic T-cell therapy delivery system of any one of claims 26-34, wherein the system selects a second cell line from the ordered set of cell lines for administration to the patient when the feedback module receives two consecutive stable disease responses, and optionally communicates the second cell line for physician approval.

36. The allogeneic T-cell therapy delivery system of claim 34, or 35, wherein the system confirms availability of the second cell line of the ordered set of cell lines in the product repository prior to communicating or shipping the second cell line.

37. The allogeneic T-cell therapy delivery system of claim 34, wherein confirming availability of the second cell line comprises confirming availability of at least three doses of the second cell line, and the system reserves two additional doses of the second cell line in conjunction with shipping a first dose of the second cell line to the patient's physician.

38. The allogeneic T-cell therapy delivery system of any one of claims 34-37, wherein a first dose of the second cell line is shipped to the physician within approximately 7 days after the feedback module receives the patient response.

39. The allogeneic T-cell therapy delivery system of any one of claims 34-38, wherein a first dose of the second cell line is shipped to the physician within approximately 6 days after the feedback module receives the patient response.

40. The allogeneic T-cell therapy delivery system of any one of claims 34-39, wherein a first dose of the second cell line is shipped to the physician within approximately 5 days after the feedback module receives the patient response.

41. The allogeneic T-cell therapy delivery system of any one of claims 34-40, wherein a first dose of the second cell line is shipped to the physician within approximately 4 days after the feedback module receives the patient response.

42. The allogeneic T-cell therapy delivery system of any one of claims 34-41, wherein a first dose of the second cell line is shipped to the physician within approximately 3 days after the feedback module receives the patient response.

43. The allogeneic T-cell therapy delivery system of any one of claims 34-42, wherein a first dose of the second cell line is administered to the patient within approximately 7 days after the feedback module receives the patient response.

44. The allogeneic T-cell therapy delivery system of any one of claims 34-43, wherein a first dose of the second cell line is administered to the patient within approximately 6 days after the feedback module receives the patient response.

45. The allogeneic T-cell therapy delivery system of any one of claims 34-44, wherein a first dose of the second cell line is administered to the patient within approximately 5 days after the feedback module receives the patient response.

46. The allogeneic T-cell therapy delivery system of any one of claims 34-45, wherein a first dose of the second cell line is administered to the patient within approximately 4 days after the feedback module receives the patient response.

47. The allogeneic T-cell therapy delivery system of any one of claims 34-46, wherein a first dose of the second cell line is administered to the patient within approximately 3 days after the feedback module receives the patient response.

48. The allogeneic T-cell therapy delivery system of any one of claims 34-47, wherein the system releases any doses of the first cell line reserved for the patient in conjunction with confirming availability of the second cell line.

49. The allogeneic T-cell therapy delivery system of any one of claims 26-48, wherein the feedback module renders a treatment complete status after administration of each cell line from the ordered set of cell lines returns a stable disease response or progressive disease response.

50. The allogeneic T-cell therapy delivery system of any one of claims 26-49, wherein the system identifies cell lines receiving a partial response from the feedback module, and outputs a notification to replenish supplies of the identified cell lines when the product repository inventory of the identified cell lines falls below a threshold level.

51. The allogeneic T-cell therapy delivery system of any one of claims 26-50, wherein the system identifies cell lines receiving a complete response from the feedback module, and outputs a notification to replenish supplies of the identified cell lines when the product repository inventory of the identified cell lines falls below a threshold level.

52. The allogeneic T-cell therapy delivery system of any preceding claim, wherein the allogeneic T-cell match generator determines an ordered set of up to four prioritized cell lines.

53. The allogeneic T-cell therapy delivery system of any preceding claim, wherein the ordered set of cell lines from the product repository consists of a single cell line.

54. The allogeneic T-cell therapy delivery system of any preceding claim, wherein the product repository is maintained at approximately −190° C. to −200° C.

55. The allogeneic T-cell therapy delivery system of any preceding claim, wherein the cell line(s) is maintained at approximately −190° C. to −200° C. during transport to the physician.

56. The allogeneic T-cell therapy delivery system of any preceding claim, wherein the cell line(s) is maintained at or below approximately −150° C. during transport to the physician.

57. The allogeneic T-cell therapy delivery system of any preceding claim, wherein the product repository is sorted by donor source.

58. The allogeneic T-cell therapy delivery system of any preceding claim, wherein the product repository is sorted by donor source HLA type.

59. The allogeneic T-cell therapy delivery system of any preceding claim, wherein the CTLs in the product repository are stored in single-use vials.

60. The allogeneic T-cell therapy delivery system of any preceding claim, wherein the patient-characteristic data includes transplant history, high resolution HLA data, and/or weight.

61. The allogeneic T-cell therapy delivery system of any preceding claim, wherein patient-characteristic data includes an HLA profile of cells from a transplanted organ.

62. The allogeneic T-cell therapy delivery system of any preceding claim, wherein patient characteristic data includes an HLA profile of transplanted allogeneic hematopoietic cells.

63. The allogeneic T-cell therapy delivery system of any preceding claim, wherein the system is configured for implementation with patients at risk, or exhibiting symptoms, of Post-Transplant Lymphoproliferative Disorder (PTLD).

64. The allogeneic T-cell therapy delivery system of any preceding claim, wherein the system is configured for implementation with patients at risk, or exhibiting symptoms, of EBV-associated Post-Transplant Lymphoproliferative Disorder (PTLD).

65. The allogeneic T-cell therapy delivery system of claim 63, wherein the PTLD is associated with a prior solid organ transplant in the patient.

66. The allogeneic T-cell therapy delivery system of claim 63, wherein the PTLD is associated with a prior hematopoietic cell transplant in the patient.

67. The allogeneic T-cell therapy delivery system of any preceding claim, wherein the system is configured for implementation with patients at risk, or exhibiting symptoms, of multiple sclerosis.

68. The allogeneic T-cell therapy delivery system of any preceding claim, wherein the system is configured for implementation with patients at risk, or exhibiting symptoms, of Cytomegalovirus infection.

69. The allogeneic T-cell therapy delivery system of any preceding claim, wherein the system is configured for implementation with patients at risk, or exhibiting symptoms, of leukemia or solid tumor cancers.

70. The allogeneic T-cell therapy delivery system of any preceding claim, wherein the antigen is a viral antigen.

71. The allogeneic T-cell therapy delivery system of claim 70, wherein the viral antigen is from Human papilloma virus.

72. The allogeneic T-cell therapy delivery system of claim 70, wherein the viral antigen is from Cytomegalovirus.

73. The allogeneic T-cell therapy delivery system of claim 70, wherein the viral antigen is from BK virus.

74. The allogeneic T-cell therapy delivery system of claim 70, wherein the viral antigen is from Epstein-Barr virus.

75. The allogeneic T-cell therapy delivery system of claim 70, wherein the viral antigen is from John Cunningham (JC) virus.

76. The allogeneic T-cell therapy delivery system of claim 70, wherein the viral antigen is from Merkel cell virus (MCV).

77. The allogeneic T-cell therapy delivery system of claim 70, wherein the antigen is a Wilm's Tumor 1 (WT1) antigen.

78. The allogeneic T-cell therapy delivery system of any preceding claim, wherein antigen-specific CTLs of the product repository meet a threshold for reactivity against cells expressing the antigen.

79. The allogeneic T-cell therapy delivery system of any preceding claim, wherein said diseased cells are tumor cells.

80. The allogeneic T-cell therapy delivery system of any preceding claim, wherein the product repository lacks CTLs having a predetermined HLA characteristic.

81. The allogeneic T-cell therapy delivery system of any preceding claim, wherein CTLs of the product repository are free of pathogenic contaminants.

82. The allogeneic T-cell therapy delivery system of any preceding claim, further including a temperature sensor to monitor the temperature of the cell line(s) throughout transport of the cell line(s).

83. The allogeneic T-cell therapy delivery system of any preceding claim, further comprising a payment module, the payment module initiating payment for the cell line(s) upon receipt of the shipment by the physician.

84. The allogeneic T-cell therapy delivery system of any preceding claim, wherein an order of the first cell line includes shipment of three separate cycles of the first cell line to the physician.

85. The allogeneic T-cell therapy delivery system of claim 84, wherein a second cycle of a first cell line is shipped approximately 7 days after the first cycle of the first cell line is shipped.

86. The allogeneic T-cell therapy delivery system of claim 85, wherein a third cycle of a first cell line is shipped approximately 7 days after the second cycle of the first cell line is shipped.

87. The allogeneic T-cell therapy delivery system of any one of claims 84-85, wherein the shipment schedule for each cycle can be adjusted by the physician.

88. A method of providing a cell therapy to a patient in need of such therapy comprising:
providing a product repository comprising a plurality of samples comprising antigen-specific Cytotoxic T-Lymphocytes (CTLs), the CTLs of each sample having a known Human Leukocyte Antigen (HLA) profile and a known HLA restriction for said antigen;
receiving patient-characteristic data, including patient identification information and an HLA profile of the patient's somatic or diseased cells;
generating an allogeneic T-cell match selection, wherein generating includes:
determining an ordered set of cell lines from the product repository, the ordered set of cell lines prioritized at least according to a pre-determined match level between the HLA profile of the patient's somatic or diseased cells and HLA profiles and known restriction(s) of cell lines in the product repository;
optionally, receiving a physician assent to treatment of the patient with the cell therapy; and
transporting the first cell line for administration to the patient.

89. The method of claim 88, further comprising storing the ordered set of cell lines in association with the patient identification information.

90. The method of claim 88 or 89, further comprising receiving a physician assent to treatment of the patient with the cell therapy.

91. The method of any one of claims 88-90, wherein the physician assent to treatment includes submission of a prescription.

92. The method of any one of claims 88-91, wherein the physician assent to treatment is received in advance of the patient's HLA profile information.

93. The method of any one of claims 88-92, wherein the physician assent to treatment is received simultaneously with the patient's HLA profile information.

94. The method of any one of claims 88-93, wherein the first cell line of the ordered set of cell lines is communicated to the patient's physician for approval.

95. The method of any one of claims 88-94, further comprising confirming current availability of a first cell line of the ordered set of cell lines in the product repository prior to communicating or transporting the first cell line to the patient's physician.

96. The method of claim 95, wherein confirming availability of the first cell line comprises confirming availability of at least three doses of the first cell line, and reserving two additional doses of the first cell line in conjunction with transporting a dose of the first cell line to the patient's physician.

97. The method of any one of claims 88-96, comprising transporting the first cell line for administration to the patient within approximately 7 days after receiving the physician assent to treatment.

98. The method of any one of claims 88-97, further comprising transporting the first cell line for administration to the patient within approximately 7 days after receiving patient characteristic data.

99. The method of any one of claims 88-98, further comprising transporting the first cell line for administration to the patient within approximately 7 days after receiving physician approval of the first cell line.

100. The method of any one of claims 88-99, wherein transporting to the physician, and/or administration to the patient, of the first cell line includes a first dose of the first cell line.

101. The method of any one of claims 88-100, further comprising providing a source repository, the source repository including donor peripheral blood mononuclear cells.

102. The method of any one of claims 88-101, further comprising contacting T lymphocytes with an antigen to form antigen-specific CTLs and storing the antigen-specific CTLs within the product repository.

103. The method of any one of claims 102, wherein the antigen is a viral antigen.

104. The method of claim 102, wherein the viral antigen is from Epstein-Barr virus.

105. The method of claim 102, wherein the viral antigen is from Human papilloma virus.

106. The method of claim 102, wherein the viral antigen is from Cytomegalovirus.

107. The method of claim 102, wherein the viral antigen is from BK virus.

108. The method of claim 102, wherein the viral antigen is from John Cunningham (JC) virus.

109. The method of claim 102, wherein the viral antigen is from Merkel cell virus (MCV).

110. The method of claim 102, wherein the antigen is a Wilm's Tumor 1 (WT1) antigen.

111. The method of any one of claims 88-109, further comprising assessing the effect of the first cell line selection, wherein assessing includes:
receiving disease response feedback from the physician;
initiating a second cycle of the first cell line when at least a partial response is provided; or
providing a next sequential cell line from the ordered set of cell lines for administration to the patient, and optionally communicating the next sequential cell line for physician approval.

112. The method of claim 110, wherein initiating a second cycle of the first cell line includes initiating a second dose.

113. The method of claim 110, wherein providing a next sequential cell line includes providing a first dose of the next sequential cell line.

114. The method of claim 110, wherein the first dose of the next sequential cell line is transported to the physician within approximately 7 days after receiving disease response feedback.

115. The method of claim 110, wherein the first dose of the next sequential cell line is transported to the physician within approximately 6 days after receiving disease response feedback.

116. The method of claim 110, wherein the first dose of the next sequential cell line is transported to the physician within approximately 5 days after receiving disease response feedback.

117. The method of claim 110, wherein the first dose of the next sequential cell line is transported to the physician within approximately 4 days after receiving disease response feedback.

118. The method of claim 110, wherein the first dose of the next sequential cell line is transported to the physician within approximately 3 days after receiving disease response feedback.

119. Theethod of any one of claims 110-118, wherein the first dose of the next sequential cell line is administered to the patient within approximately 7 days after receiving disease response feedback.

120. The method of any one of claims 110-119, wherein the first dose of the next sequential cell line is administered to the patient within approximately 6 days after receiving disease response feedback.

121. The method of any one of claims 110-120, wherein the first dose of the next sequential cell line is administered to the patient within approximately 5 days after receiving disease response feedback.

122. The method of any one of claims 110-121, wherein the first dose of the next sequential cell line is administered to the patient within approximately 4 days after receiving disease response feedback.

123. The method of any one of claims 110-122, wherein the first dose of the next sequential cell line is administered to the patient within approximately 3 days after receiving disease response feedback.

124. The method of any one of claims 88-123, wherein generating an allogeneic T-cell match selection includes generating up to four prioritized cell lines.

125. The method of any one of claims 88-124, further comprising expanding the antigen-specific CTLs.

126. The method of any one of claims 88-125, further comprising assessing allo-reactivity of the antigen-specific CTLs and excluding or discarding CTLs exhibiting allo-reactivity above a predetermined threshold prior to adding the cells to the product repository.

127. The method of any one of claims 88-126, further comprising assessing the anti-antigen reactivity of the antigen-specific CTLs, and adding them to the product repository if the CTLs are determined to meet a threshold for anti-antigen reactivity.

128. The method of any one of claims 88-127, further comprising evaluating the HLA profile of the antigen-specific CTLs of the product repository and storing the HLA profile in connection with each sample of CTLs.

129. The method of any one of claims 88-128, further comprising administering the cell therapy to a patient at risk, or exhibiting symptoms, of Post-Transplant Lymphoproliferative Disorder (PTLD).

130. The method of any one of claims 88-128, further comprising administering the cell therapy to a patient at risk, or exhibiting symptoms, of EBV-associated Post-Transplant Lymphoproliferative Disorder (PTLD).

131. The method of any one of claims 88-127, further comprising administering the cell therapy to a patient at risk, or exhibiting symptoms, of multiple sclerosis.

132. The method of any one of claims 88-127, further comprising administering the cell therapy to a patient at risk, or exhibiting symptoms, of leukemia, or a solid tumor cancer.

133. The method of any one of claims 88-127, further comprising administering the cell therapy to a patient at risk, or exhibiting symptoms, of CMV infection.

134. The method of any one of claims 88-127, further comprising administering the cell therapy to a patient at risk, or exhibiting symptoms, of a hematological disorder characterized by expression of a Wilm's Tumor 1 antigen.
